(19) United States
(12) Patent Application Publication
(10) Pub. No.: US 2009/0250070 A1
(43) Pub. Date: Oct. 8, 2009

(54) Apparatus and Method for Influencing the Sleep of a Person, Operating Unit and Computer Program Product

(75) Inventor: Thomas Pfeifer, Frankfurt am Main (DE)

Correspondence Address: Maginot, Moore & Beck;Chase Tower, Suite 3250, 111 Monument Circle, Indianapolis, IN, 46204, US

(73) Assignee: Dr. Thomas Pfeifer, Frankfurt am Main (DE)

(21) Appl. No.: 12/416,613

(22) Filed: Apr. 1, 2009

(30) Foreign Application Priority Data
Apr. 2, 2008 (DE) 10 2008 016855.6

(51) Int. Cl. A61F 5/56

(52) U.S. Cl. 128/848

(57) ABSTRACT

The invention develops an apparatus and a method for influencing the sleep of a person. The apparatus comprises a sensor device for detecting a current rotational position of the body of the person about the longitudinal axis of the body, an actuator device for supplying a predeterminable stimulus pattern to the body, and an operating unit with a selection device for selecting at least one desired and/or at least one prohibited rotational position of the body of the person about the longitudinal axis of the body, a comparator device for comparing the current rotational position and the at least one desired and/or at least one prohibited rotational position and emitting a corresponding position signal, and a control device which can be set to actuate the actuator device to supply the predeterminable stimulus pattern to the body in response to the position signal if the position signal shows that the current rotational position does not correspond to the at least one desired rotational position and/or corresponds to at least the one prohibited rotational position.

### BACKGROUND

The present invention relates to an apparatus and a method for influencing the sleep of a person, an operating unit and a computer program product.

DE 101 14 205 B1 discloses an apparatus for correcting the sleep posture of a person, which comprises an attachment device, which can be attached to the body of the person as an abdominal belt, and comprises at least one sensor device, which is attached to the attachment device, for determining a prone position or a region about the prone position as a predetermined rotational position of the body of the person. An actuator device attached to the attachment device transmits a predeterminable dermal stimulus to the body of the person in the region of the attachment device, responding to the sensor device detecting that the rotational position is in the prone position or in the region about the prone position.

FIG. 7 is a block diagram of an apparatus, disclosed in DE 101 14 205 B1, for correcting the sleep posture of a person.

In the schematic diagram shown in FIG. 7, a body 100 of a human being is arranged whilst sleeping on a support 102 in a preferably horizontal position, i.e. a longitudinal axis of the body 104 is aligned parallel to the horizontally aligned support 102, with an initial sleeping position being a supine position. A sensor-actuator module 103 is attached to the body 100 of the human being by means of an attachment device 101 which can be designed as an abdominal belt for example.

The attachment device 101 can resist movements of the body 100. Moreover, attaching the attachment device 101 to the body 100 of the human being only has minor adverse effects on the sleep of the human being because, in principle, movements of the body 100 in all directions are allowed, in particular a rotational movement of the body 100 of a human being about the longitudinal axis of said body 104.

FIG. 8 is a flowchart which illustrates the procedure of a method for correcting the sleep posture of a person using the known apparatus in accordance with FIG. 7.

After starting up the apparatus according to the invention in step 401, the position is firstly determined in step 402. The output signals of the sensor device are evaluated in step 403 with respect to a position, in particular a rotational position about a longitudinal axis of the body 100.

Step 404 determines whether or not the determined position is proper with respect to a predetermined target position (proper=supine position, lateral position, . . . ). If it is determined in step 404 that the position is proper, then in step 406 a predetermined time, a so-called dwell time, is waited for, until the processing returns to step 402.

Subsequently, the steps 402, 403 and 404 are rerun. If it is determined in step 404 that the position is not proper (not proper=prone position) then activation of the actuator device is initiated in step 405. A stimulus output of the actuator devices leads to stimulation of the body 100 of a human being by means of a piezo-actuator, a heating element, a cooling element, vibrations, etc., as described above so that the sleeping human being changes his/her sleeping position as a result of this stimulus. After the actuator is activated in step 405, the duration and intensity of which can be predetermined by the processing unit, the processing returns to step 402, where the position is once again determined. The processing in turn runs through steps 402, 403 and 404, until it is determined in step 404 that the attained position is proper.

If it is determined in step 404 that the position is still not proper, the processing once again proceeds with step 405 and subsequently step 402.

Otherwise, if it is determined in step 404 that the position is proper, the processing advances to step 406 and, after a dwell time, to step 402. A dwell time, set in step 406, is selected such that the position is only determined at particular points in time, which can be predetermined by the processing unit 304, so that too frequent stimulation of the body 100 of a human being, and thus the risk of waking up, is avoided.

Furthermore, a predeterminable dwell time is used to reduce the energy requirements of the overall arrangement and keep the load on the energy supply unit 305 minimal.

These known apparatuses make it possible to avoid undesirable pressure and/or tension which lead or leads to blockages in the region of the vertebral joints, in particular the cervical vertebral joints, which are known by the name of “torticollis” (acute wry neck).

DE 83 28 640 U1 discloses an apparatus for correcting the sleep posture of a person, in which an ear button, which can be worn in the ear of the user, is provided with a signal generator which can be initiated by a change in position.

### SUMMARY

It is an object of the present invention to develop an apparatus and a method for influencing the sleep of a person, an operating unit and a computer program product which can be used in a more flexible manner.

The invention develops an apparatus for influencing the sleep of a person, a method for influencing the sleep of a person, an operating unit, and a computer program product.

The subject matter according to the invention has many advantages. In particular, is allows for flexible use in various therapies.

The dependent claims comprise advantageous developments and improvements of the respective subject matter of the invention.

According to one preferred development, provision is made for a second sensor device for detecting snoring and for emitting a corresponding snore signal, it being possible for the control device to be set by a second selection device provided in the operating unit,
to actuate the actuator device to supply the predeterminable stimulus pattern to the body in response to the snore signal if the snore signal shows that the person is snoring and/or
to actuate the actuator device to supply the predeterminable stimulus pattern to the body in response to the position signal if the position signal shows that the current rotational position does not correspond to the at least one desired rotational position and/or corresponds to at least the one prohibited rotational position.

According to a further preferred development, provision is made for a third sensor device for detecting a circulation parameter of the person and for emitting a corresponding circulation signal, it being possible for the control device to be set by the second selection device,
to actuate the actuator device to supply the predeterminable stimulus pattern to the body in response to the circulation signal if the circulation signal shows that there is a disturbance in the circulation of the person and/or
to actuate the actuator device to supply the predeterminable stimulus pattern to the body in response to the position signal if the position signal shows that the current rotational position does not correspond to the at least one desired rotational position and/or corresponds to at least the one prohibited rotational position and/or
to actuate the actuator device to supply the predeterminable stimulus pattern to the body in response to the snore signal if the snore signal shows that the person is snoring.

According to a further preferred development, provision is made for a third selection device in the operating unit for selecting the stimulus pattern.

According to a further preferred development, the third selection device is designed such that different stimulus patterns can be selected corresponding to the presence of different combinations of position signal, snore signal and circulation signal, which signals cause the control device to actuate the actuator device.

According to a further preferred development, the second sensor device has a microphone. It is preferable for the bandwidth of the microphone to be specific to snoring and it preferably lies between 30 and 1600 Hz.

According to a further preferred development, the sensitivity and/or frequency characteristic of the second sensor device can be adjusted.

According to a further preferred development, the third sensor device has a heart rate sensor and/or a blood oxygen saturation sensor.

According to a further preferred development, the heart rate and/or the blood oxygen saturation which indicate that there is a disturbance in the circulation of the person can be adjusted.

According to a further preferred development, provision is made for a dwell time element which is designed to interrupt operation of the apparatus in a predeterminable manner.

### BRIEF DESCRIPTION OF THE DRAWINGS

Exemplary embodiments of the invention are explained in more detail in the description below and are illustrated in the drawings, in which:

FIG. 1 shows a block diagram of a first embodiment of the apparatus for influencing the sleep of a person according to the invention;

FIG. 2 shows a flowchart illustrating the procedure of a method for influencing the sleep of a person using the apparatus in accordance with the first embodiment;

FIG. 3 shows a block diagram of a second embodiment of the apparatus for influencing the sleep of a person according to the invention;

FIG. 4 shows a flowchart illustrating the procedure of a method for influencing the sleep of a person using the apparatus in accordance with the second embodiment;

FIG. 5 shows a block diagram of a third embodiment of the apparatus for influencing the sleep of a person according to the invention;

FIG. 6 shows a flowchart illustrating the procedure of a method for influencing the sleep of a person using the apparatus in accordance with the third embodiment;

FIG. 7 shows a block diagram of a known apparatus for correcting the sleep posture of a person disclosed in DE 101 14 205 B1; and

FIG. 8 shows a flowchart illustrating the procedure of a method for correcting the sleep posture of a person using the known apparatus in accordance with FIG. 7.

### DETAILED DESCRIPTION

In the figures, identical or functionally identical components are designated by the same reference symbol.

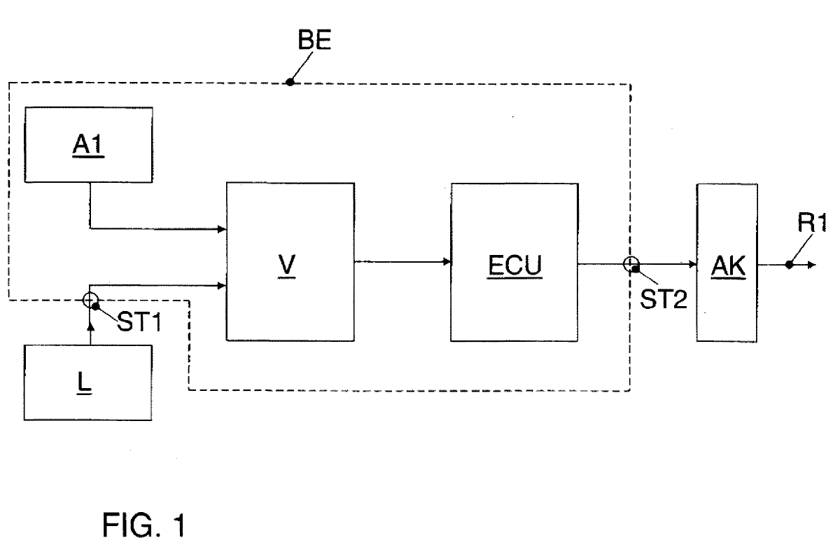

FIG. 1 is a block diagram of a first embodiment of the apparatus for influencing the sleep of a person according to the invention.

In FIG. 1, reference symbol BE designates an operating unit for an apparatus for influencing the sleep of a person, for example standalone equipment or equipment that can be attached to a person. The operating unit BE has a first connection ST1 for connecting a sensor device L, which can be attached to a person, for detecting a current rotational position of the body of the person about the longitudinal axis of the body. Furthermore, the operating unit BE has a second connection ST2 for connecting an actuator device AK for supplying a predeterminable stimulus to the body, for example a vibrational stimulus.

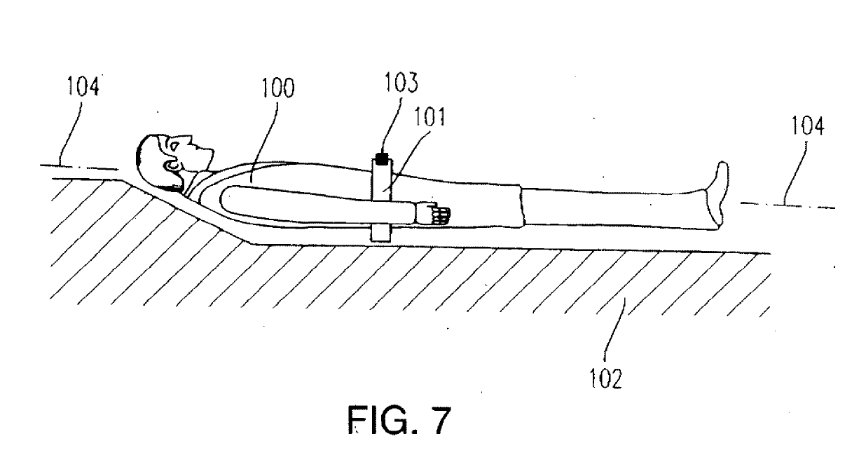

A corresponding sensor device L is connected to the first connection ST1 and a corresponding actuator device AK is connected to the second connection ST2, both of which are designed in an abdominal belt in the present example, that is to say they are attached to the body 100 of the human being as a sensor-actuator module 103 (cf. FIG. 7).

A first selection device A1, for selecting at least one desired and/or at least one prohibited rotational position of the body of the person about the longitudinal axis of the body, is housed in the operating unit BE. Such a first selection device A1 can, for example, be present in the form of a display with an associated set-point adjustment device. In simple embodiments, it can also be a simple rotary knob or a number of rotary knobs when setting a rotational position range.

The predetermined desired and/or prohibited rotational positions, and the current rotational position determined from the first sensor device L, are fed into a comparator device V located in the operating unit BE.

The comparator device V compares the current rotational position and the desired and/or prohibited rotational position and emits a corresponding position signal to a control device ECU which is likewise located in the operating unit BE.

The control device ECU actuates the actuator device AK for supplying the predeterminable stimulus pattern R1 to the body in response to the position signal if the position signal shows that the current rotational position does not correspond to the desired rotational positions and/or corresponds to the prohibited rotational positions.

By way of example, the first sensor device L can be an optical sensor which operates on the basis of the Sagnac effect by providing a fibre-gyroscopic device by means of which rotations of the body about the longitudinal axis can be detected.

Hence, the apparatus according to the first embodiment can be used to define any rotational position of the body of the person about the longitudinal axis of the body as either desired or prohibited. Thus, in contrast to the prior art, it is possible in practical applications to not only prevent prone positions but in particular supine positions, which can be dangerous for snoring patients, as well. It is also possible to avoid lateral positions in the case of specific injuries.

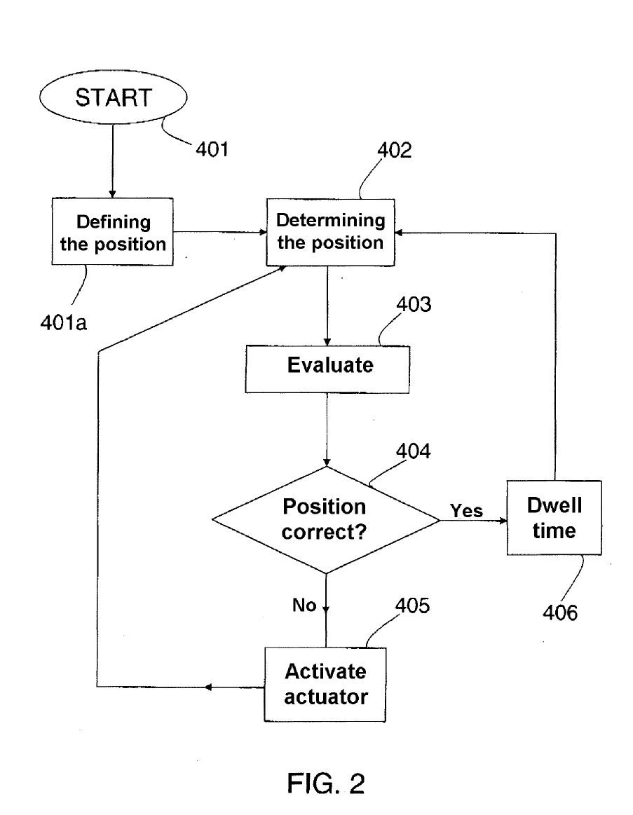

FIG. 2 shows a flowchart illustrating the procedure of a method for influencing the sleep of a person using the apparatus in accordance with the first embodiment.

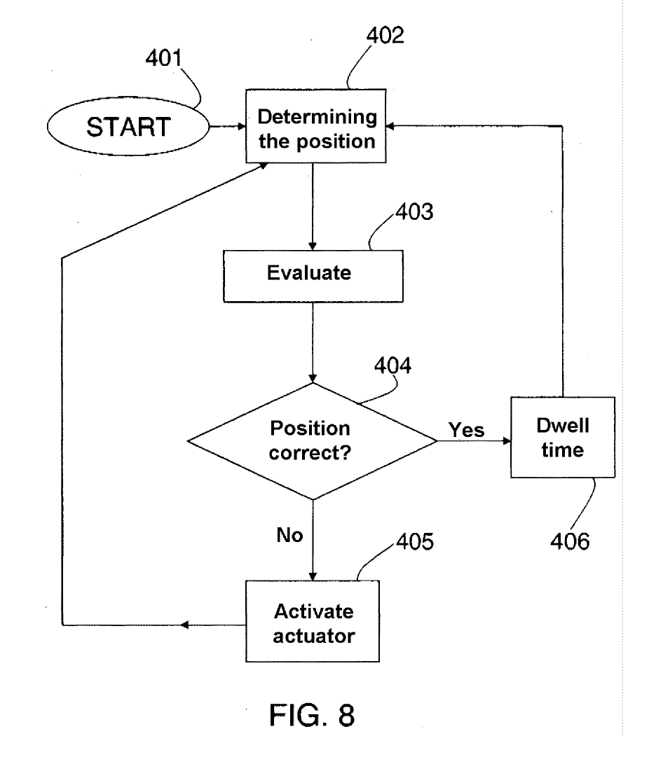

Unlike the known method explained above in conjunction with FIG. 8, the method in accordance with FIG. 2 comprises an additional step 401a which is referred to as defining the position. This step 401a is carried out immediately after the start 401, to be precise before the determining of the position in step 402 is commenced.

In step 401a, the user uses the first selection device A1 to input the desired and/or the prohibited rotational positions or positions, and optionally the associated tolerance ranges as well.

The subsequent determining of the position in step 402 and the further steps 403, 404, 405, 406 are carried out as already explained in great detail in conjunction with FIG. 8.

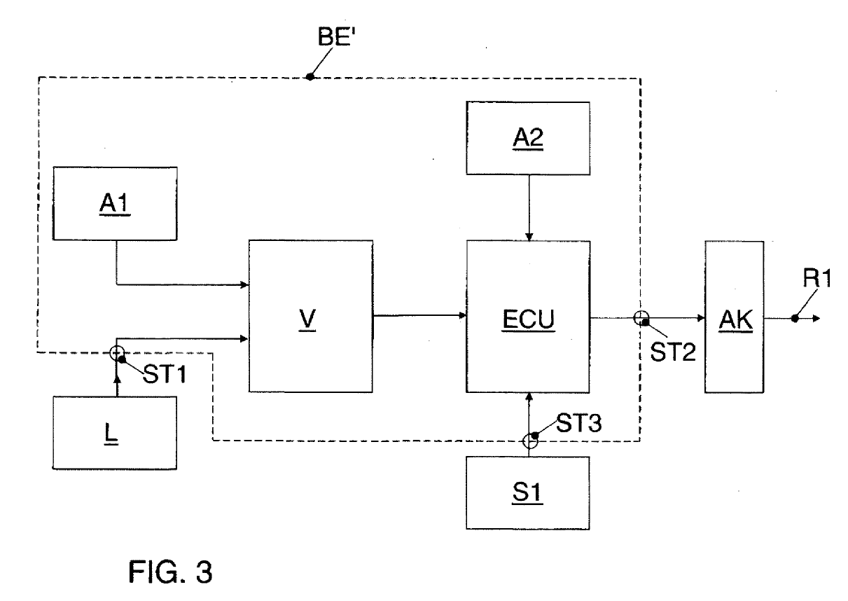

FIG. 3 is a block diagram of a second embodiment of the apparatus for influencing the sleep of a person according to the invention.

In the second embodiment in accordance with FIG. 3, the reference symbol BE′ designates an operating unit which is extended compared to the operating unit BE in accordance with the first embodiment.

In particular, the operating unit BE′ comprises a third connection ST3 for connecting a second sensor device S1 for detecting snoring and for emitting a corresponding snore signal to the control device ECU. The second sensor device S1 can, for example, have a microphone, the bandwidth of which is preferably specific to snoring and for example lies between 30 and 1600 Hz. Optionally, the sensitivity and/or the frequency characteristic of this microphone can be adjustable in order to allow fine-tuning appropriate to the respective snoring patient.

Furthermore, the operating unit BE′ additionally has a second selection device A2, as a result of which the control device ECU can be set to either react only to the output signal of the comparator device V or to react only to the snore signal of the second sensor device S1 in the form of the microphone, or even to react only if both the output signal of the comparator device V and the snore signal require intervention by the actuator device AK.

In other words, the second selection device A2 makes it possible to fix whether actuator intervention should already occur on the basis of the fact that the rotational position is incorrect, or already on the basis of the fact that snoring is present, or only if snoring occurs in an undesired rotational position, i.e. both intervention conditions must be satisfied. In the case of a snoring patient, the supine position is undesirable; here, the lower jaw and the tongue can fall backwards due to gravity and this can lead to narrowing of the air passages or to a complete collapse—obstructive sleep apnea—and this affects 10% of people who snore.

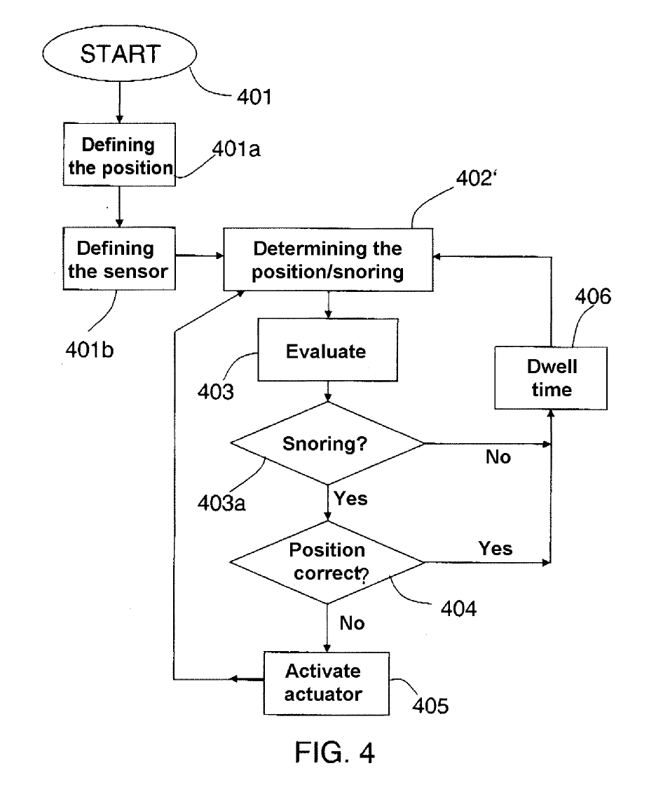

FIG. 4 is a flowchart illustrating the procedure of a method for influencing the sleep of a person using the apparatus in accordance with the second embodiment.

In the method illustrated in FIG. 4, there is, in addition to the method described in conjunction with FIG. 2, a step 401b in which a sensor is defined by means of the second selection device A2, as explained previously. Using the selection device A2, which is present for example in the form of a rotary switch or any other type of switch, it is thus possible to fix the first sensor device L or the second sensor device S1 or the first and the second sensor device L, S1 as being decisive for determining an intervention of the actuator device AK by the control device ECU.

If, as is the case in the present exemplary embodiment, both the first sensor device L and the second sensor device S1 have been selected as being decisive for an intervention, the position and the snoring are determined in step 402′. After the evaluation step 403, there is step 403a, in which it is determined whether the second sensor device S1 shows that snoring is present. If this is not the case, the program branches to step 406, in which the dwell time element is passed, and then returns again to step 402′ for determining the position and the snoring.

If snoring is present in step S403a, the program continues to step 404, where a check is made as to whether the position is proper, to be precise on the basis of the defined positions set in step 401a. If the position is proper, the program likewise branches to step 406, in which the dwell time element is passed, and then returns again to step 402′ for determining the position and the snoring.

If both conditions are met, that is to say that both snoring was detected in step 403a and an improper position was detected in step 404, then the actuator is activated in step 405, for example in the form of initiating the vibration of the vibration device corresponding to the stimulus pattern R1, in order to move the patient to turn to another position, for example the prone position.

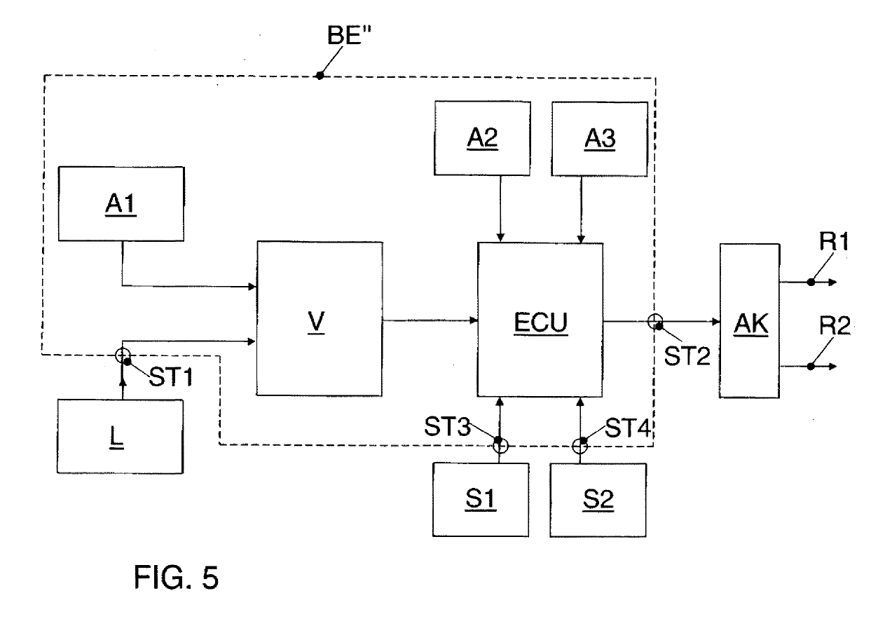

FIG. 5 is a block diagram of a third embodiment of the apparatus for influencing the sleep of a person according to the invention.

In the case of the third embodiment in accordance with FIG. 5, the operating unit is designated by the reference symbol BE″. In addition to the components of the operating unit BE′, the operating unit BE″ has a fourth connection ST4 for connecting a third sensor device S2, the third sensor device S2 in the current example being a blood oxygen saturation sensor which measures the blood oxygen concentration in the blood of the patient and emits a corresponding blood oxygen signal as a circulation signal.

The control device ECU receives the blood oxygen signal from the second sensor device S2 which can be set by the second selection device A2 to actuate the actuator device AK to supply a predeterminable stimulus pattern in response to the blood oxygen signal if the blood oxygen signal shows that the circulation of the person is disturbed.

For example, a decrease in the oxygen saturation by 4% can constitute a critical situation for the patient, and so this justifies such an intervention. Optionally, such a critical value of the oxygen saturation can be predetermined or a corresponding limiting value can be set in the second sensor device S2.

As is the case in the second embodiment, the second selection device A2 can fix whether the control device ECU reacts, that is to say initiates an actuator intervention, only to the position signal, only to the snore signal, only to the blood oxygen signal or to any combination thereof.

Furthermore, the operating unit BE2′ provides a third selection device A3, by means of which it is possible to fix which stimulus pattern R1, R2 is initiated in accordance with the presence of different combinations of position signal, snore signal and blood oxygen signal. For example, in the present example, it has been decided that in the case where snoring and an improper position are present, but the blood oxygen signal is correct, a stimulus R1, for example said vibration, is emitted.

In contrast, if the blood oxygen concentration is too low in addition to the snoring and an improper position, then the selection device 3 makes it possible to set in advance that a second stimulus pattern R2 is emitted which, for example, effects a stronger intervention on the patient, for example a waking-up signal from an acoustic waking-up device or a stronger vibrational stimulus, the second actuator signal being intended to ensure that the patient definitely wakes up or changes position.

The third selection device A3 can also be present in the form of a rotary switch or any other type of switch. The signal emitted by the control device ECU via the second input ST2 lets the actuator device AK know which specific actuator is intended to be activated, for example vibrations for the stimulus pattern R1 and/or an alarm for the stimulus pattern R2.

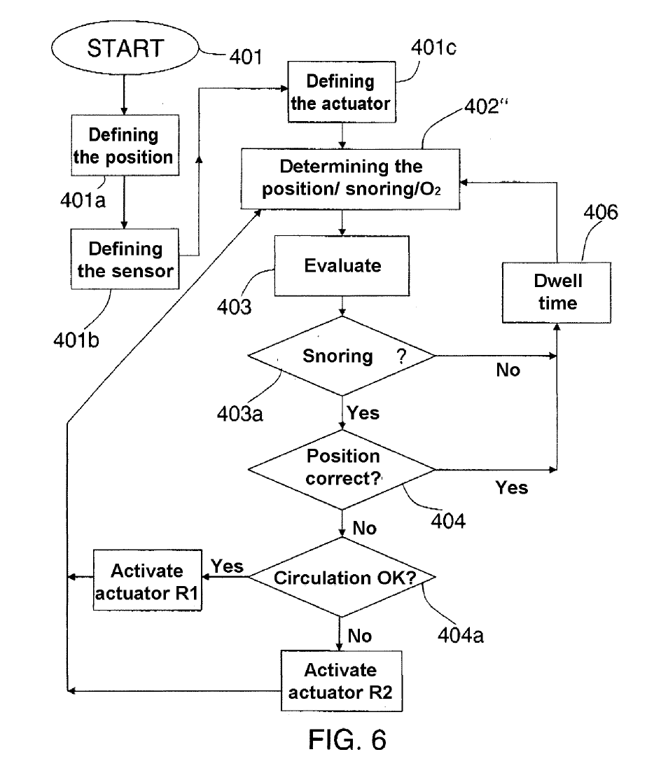

FIG. 6 is a flowchart illustrating the procedure of a method for influencing the sleep of a person using the apparatus in accordance with the third embodiment.

In the method illustrated in FIG. 6, there is, in addition to the method described in conjunction with FIG. 4, the step 401c in which the actuator is defined before detecting the position/snore/O₂ in step 402″ and said defining of the actuator determining which actuator device is intended to be active for which sensor criteria. That is to say, in this example improper position and additionally snoring is defined to initiate the stimulus pattern R1 and the stimulus pattern R2 is carried out if additionally the circulation is disturbed according to the blood oxygen signal.

Furthermore, if steps 403a and 404 were answered with YES, then the step 404a is carried out after step 404, in which it is determined whether the blood oxygen value is alright.

If this is the case, then the actuator device AK is initiated to emit the stimulus pattern R1, which means commencing vibrations in the present case.

If it is determined in step 404a that the circulation is not alright, that is to say that a too low blood oxygen saturation is present, then the control device ECU causes the actuator device AK to emit the stimulus pattern R2 which is a loud waking-up signal in the present case and is intended to ensure that the patient wakes up.

Although the present invention was described above on the basis of preferred exemplary embodiments, it is not limited thereto; rather it is possible to modify the invention in a number of ways.

In particular, the invention is not limited to the sensor/actuator combinations described above; rather it can be applied to any sensor/actuator combination.

1. Apparatus for influencing the sleep of a person comprising:
a) a first sensor device which can be attached to the person for detecting a current rotational position of a body of the person about the longitudinal axis of the body;
b) an actuator device for supplying a predetermined stimulus pattern to the body; and
c) an operating unit with
c1) a first selection device for selecting at least one desired and/or at least one prohibited rotational position of the body of the person about the longitudinal axis of the body;
c2) a comparator device for comparing the current rotational position and the at least one desired and/or at least one prohibited rotational position and providing a corresponding position signal; and
c3) a control device which can be set to actuate the actuator device to supply the predetermined stimulus pattern to the body in response to the position signal if the position signal shows that the current rotational position does not correspond to the at least one desired rotational position and/or corresponds to at least the one prohibited rotational position.

2. Apparatus for influencing the sleep of a person according to claim 1, further comprising a second sensor device for detecting snoring and for providing a corresponding snore signal, it being possible for the control device to be set by a second selection device provided in the operating unit,
to actuate the actuator device to supply the predeterminable stimulus pattern to the body in response to the snore signal if the snore signal shows that the person is snoring and/or
to actuate the actuator device to supply the predeterminable stimulus pattern to the body in response to the position signal if the position signal shows that the current rotational position does not correspond to the at least one desired rotational position and/or corresponds to at least the one prohibited rotational position.

3. Apparatus for influencing the sleep of a person according to claim 1, further comprising a third sensor device for detecting a circulation parameter of the person and for emitting a corresponding circulation signal, it being possible for the control device to be set by the second selection device,
to actuate the actuator device to supply the predeterminable stimulus pattern to the body in response to the circulation signal if the circulation signal shows that there is a disturbance in the circulation of the person and/or
to actuate the actuator device to supply the predeterminable stimulus pattern to the body in response to the position signal if the position signal shows that the current rotational position does not correspond to the at least one desired rotational position and/or corresponds to at least the one prohibited rotational position and/or
to actuate the actuator device to supply the predeterminable stimulus pattern to the body in response to the snore signal if the snore signal shows that the person is snoring.

4. Apparatus for influencing the sleep of a person according to claim 2, further comprising a third sensor device for detecting a circulation parameter of the person and for emitting a corresponding circulation signal, it being possible for the control device to be set by the second selection device,
to actuate the actuator device to supply the predeterminable stimulus pattern to the body in response to the circulation signal if the circulation signal shows that there is a disturbance in the circulation of the person and/or
to actuate the actuator device to supply the predeterminable stimulus pattern to the body in response to the position signal if the position signal shows that the current rotational position does not correspond to the at least one desired rotational position and/or corresponds to at least the one prohibited rotational position.

5. Apparatus for influencing the sleep of a person according to claim 1, further comprising by a third selection device provided in the operating unit for selecting the stimulus pattern.

6. Apparatus for influencing the sleep of a person according to claim 4, further comprising by a third selection device provided in the operating unit for selecting the stimulus pattern, and wherein the third selection device is designed such that different stimulus patterns can be selected corresponding to the presence of different combinations of position signal, snore signal and circulation signal, which signals cause the control device to actuate the actuator device.

7. Apparatus for influencing the sleep of a person according to claim 1, wherein an attachment device which can be attached to the body of the person in the form of an abdominal belt and onto which the first sensor device and the actuator device can be fixed.

8. Apparatus for influencing the sleep of a person according to claim 1, wherein the actuator device is designed to generate an acoustic stimulus pattern and/or a vibrational stimulus pattern.

9. Apparatus for influencing the sleep of a person according to claim 1, wherein the first sensor device has an optical sensor operating on the basis of the Sagnac effect by providing a fibre-gyroscopic device by means of which rotations of the body about the longitudinal axis of the body can be detected.

10. Apparatus for influencing the sleep of a person according to claim 2, wherein the second sensor device has a microphone.

11. Apparatus for influencing the sleep of a person according to claim 10, wherein the bandwidth of the microphone is specific to snoring and preferably lies between 30 and 1600 Hz.

12. Apparatus for influencing the sleep of a person according to claim 2, wherein the sensitivity and/or frequency characteristic of the second sensor device can be adjusted.

13. Apparatus for influencing the sleep of a person according to claim 3, wherein the third sensor device has a heart rate sensor and/or a blood oxygen saturation sensor.

14. Apparatus for influencing the sleep of a person according to claim 12, wherein the heart rate and/or the blood oxygen saturation which indicate that there is a disturbance in the circulation of the person can be adjusted.

15. Apparatus for influencing the sleep of a person according to claim 1, wherein a dwell time element is provided which is designed to interrupt operation of the apparatus in a predetermined manner.

16. Method for influencing the sleep of a person comprising the steps of:
a) detecting a current rotational position of a body of the person about the longitudinal axis of the body;
b) selecting at least one desired and/or at least one prohibited rotational position of the body of the person about the longitudinal axis of the body by means of an operating unit;
c) supplying a predeterminable stimulus pattern to the body;
d) comparing the current rotational position and the at least one desired and/or at least one prohibited rotational position and emitting a corresponding position signal in the operating unit; and
f) actuating the actuator device for supplying the predeterminable stimulus pattern to the body by the operating unit if the position signal shows that the current rotational position does not correspond to the at least one desired rotational position and/or corresponds to at least the one prohibited rotational position.

17. Operating unit for carrying out the method according to claim 16, comprising
a first connection for connecting a first sensor device, which can be attached to the person, for detecting a current rotational position of the body of the person about the longitudinal axis of the body;
a first selection device for selecting at least one desired and/or at least one prohibited rotational position of the body of the person about the longitudinal axis of the body;
a second connection for connecting an actuator device for supplying a predeterminable stimulus pattern to the body;
a comparator device for comparing the current rotational position and the at least one desired and/or at least one prohibited rotational position and emitting a corresponding position signal; and
a control device which can be set to actuate the actuator device to supply the predeterminable stimulus pattern to the body in response to the position signal if the position signal shows that the current rotational position does not correspond to the at least one desired rotational position and/or corresponds to at least the one prohibited rotational position.

18. Computer program product with a machine-readable code, by means of which the method according to claim 16 can be executed on an operating unit.
